(19) United States
(12) Patent Application Publication
(10) Pub. No.: US 2026/0125648 A1
(43) Pub. Date: May 7, 2026

(54) LYOPHILIZATE POWDER OF STEM CELLS FROM CERVIDS, PREPARATION METHOD THEREOF, USE THEREOF AND COMPOSITION THEREOF

(71) Applicant: U-NEURON BIOMEDICAL INC., New Taipei City (TW)

(72) Inventors: Hsiang-Hung Huang, New Taipei City (TW); Yu-Hsin Chu, New Taipei City (TW); Tzu-Hao Huang, New Taipei City (TW); Yen-Hua Chiu, New Taipei City (TW); Hsiao-Yun Li, New Taipei City (TW)

(73) Assignee: U-NEURON BIOMEDICAL INC., New Taipei City (TW)

(21) Appl. No.: 19/378,289

(22) Filed: Nov. 3, 2025

(30) Foreign Application Priority Data
Nov. 4, 2024 (TW) 113142143

(51) Int. Cl. C12N 5/0775; A61K 31/198; A61K 35/28; A61K 38/18; C12N 5/00

(52) U.S. Cl. CPC C12N 5/0668; A61K 31/198; A61K 35/28; A61K 38/1841; C12N 5/562; C12N 2509/10

(57) ABSTRACT

Lyophilizate powder of stem cells from cervids includes lyophilized powder from stem cells of an antler and lyophilized powder from a conditioned medium. The lyophilized powder from stem cells of an antler includes whole cell lyophilized powder from whole cell components, and the conditioned medium is the conditioned medium after culturing the stem cells of an antler.

### BACKGROUND OF THE INVENTION

### 1. Field of the Invention

The present disclosure generally relates to a lyophilized powder of stem cells derived from a cervid animal, a method for preparing the lyophilized stem cell powder of a cervid animal, a use thereof, and a composition comprising a lyophilized powder obtained from cervid stem cells. More particularly, the present disclosure pertains to a lyophilized stem cell powder of a cervid animal that comprises a lyophilized powder of deer antler stem cells and a lyophilized powder of a conditioned medium, a method for preparing such a lyophilized stem cell powder, a use thereof, and a composition comprising the lyophilized stem cell powder of the cervid animal.

### 2. Description of the Prior Art

The term deer antler refers to the immature antler of a male deer before ossification, when the antler remains covered with velvet hair and has not yet hardened into bone. Deer antler is regarded as a valuable traditional Chinese medicinal material. According to ancient literature, such as the Compendium of Materia Medica (Ben Cao Gang Mu), deer antler is described to have the effect comprising: nourish the marrow, enrich the blood, strengthen tendons and bones, treat ebilitation, hearing loss, dim vision, dizziness, and chronic diarrhea.” Owing to its remarkable efficacy, deer antler has long been used in traditional Chinese formulations, not only for the above-mentioned effects but also for promoting metabolism, maintaining a youthful appearance, improving sleep quality, and reducing fatigue. Consequently, deer antler has been highly esteemed among East Asian populations and has been widely used for generations in regions such as China, Japan, Korea, and Siberia.

In recent years, studies have reported that deer antler contains high concentrations of growth factors, including insulin-like growth factor type 1 (IGF-1) and insulin-like growth factor type 2 (IGF-2), as well as other growth factors that promote the activation of epidermal cells, neuronal cells, and epithelial cells. These bioactive factors are believed to account for the rapid annual regrowth of deer antlers. Furthermore, deer antler is rich in amino acids and various cofactors such as glucosamine, chondroitin, phospholipids, hypoxanthine, uracil derivatives, prostaglandins, and polyamines. It also contains complex vitamins, lecithin, and multiple minerals, such as magnesium, phosphorus, calcium, sodium, and potassium, as well as trace elements such as selenium, zinc, silicon, manganese, copper, and aluminum.

However, the acquisition of deer antler is relatively difficult, as stem cells possessing differentiation potential can only be obtained during the early growth stage of the antler. Therefore, how to effectively utilize the precious bioactive components within deer antler to meet consumer demand has become a key challenge and direction for industrial development.

### SUMMARY OF THE INVENTION

In view of the drawbacks encountered in existing products during practical application, the present disclosure provides a lyophilized stem cell powder of a cervid animal, with improved efficacy in promoting osteogenic differentiation, anti-inflammatory activity, and antioxidant activity.

The present disclosure first provides a lyophilized stem cell powder of a cervid animal, wherein the lyophilized stem cell powder comprises a lyophilized powder of deer antler stem cells and a lyophilized powder of a conditioned medium.

The lyophilized stem cell powder of the deer antler contains a whole-cell lyophilized powder including all cellular components, and the conditioned medium is obtained after culturing the deer antler stem cells.

In at least one embodiment, the deer antler stem cells express at least one antigen selected from the group consisting of CD73, CD90, and CD105.

In at least one embodiment, the protein content of the lyophilized stem cell powder of the cervid animal ranges from about 101.2 mg/g to 435.2 mg/g.

In at least one embodiment, the lyophilized stem cell powder of the cervid animal comprises: 1.12 mg/g to 3.32 mg/g of essential amino acids; 0.35 mg/g to 1.54 mg/g of branched-chain amino acids; or 2.71 mg/g to 6.03 mg/g of total amino acids.

In at least one embodiment, the activin (Activin) content of the lyophilized stem cell powder of the d cervid animal ranges from about 12.1 ng/g to 155.3 ng/g.

In at least one embodiment, the lyophilized stem cell powder of the cervid animal contains no viable cells.

In at least one embodiment, the lyophilized stem cell powder of the cervid animal further comprises a cosmetically or pharmaceutically acceptable bulking agent.

The present disclosure further provides a lyophilized powder composition comprising the lyophilized stem cell powder and a promoting agent that enhances at least one of osteogenic differentiation, anti-inflammatory activity, and antioxidant activity.

The present disclosure further provides a method for preparing the lyophilized stem cell powder of a cervid animal, comprising: culturing deer antler stem cells to obtain a conditioned medium; collecting the conditioned medium and the cultured deer antler stem cells; and subjecting both the collected conditioned medium and the collected deer antler stem cells to freeze-drying and pulverization to obtain the lyophilized stem cell powder.

In at least one embodiment, prior to lyophilization and pulverization, the collected conditioned medium and deer antler stem cells are mixed to form a sample.

In at least one embodiment, the sample is lyophilized by condensing at −40° C., vacuuming to a pressure of 0.2 torr, heating to 30° C., and sublimating, thereby obtaining a lyophilized stem cell product of a cervid animal.

In at least one embodiment, the lyophilized stem cell product of the cervid animal is pulverized using a grinder and sieved through a 0.6 mm mesh to obtain the lyophilized stem cell powder of the cervid animal.

In at least one embodiment, the conditioned medium is a DMEM culture medium.

The present disclosure further provides a use of the lyophilized stem cell powder of a cervid animal for preparing a lyophilized powder composition that promotes at least one of osteogenic differentiation, anti-inflammatory activity, and antioxidant activity.

The present disclosure further provides a method for alleviating, treating, or preventing a medical condition associated with bone formation, inflammation, or oxidation, comprising administering to a subject in need thereof either the lyophilized stem cell powder of a cervid animal alone, or in combination with at least one promoting agent that enhances osteogenic differentiation, anti-inflammatory activity, and/or antioxidant activity.

In at least one specific embodiment of the present disclosure, the deer antler stem cells are purified and banked as a cell stock, followed by expansion of the deer antler stem cells and their conditioned medium. Both the cultured cells and the conditioned medium are collected and lyophilized to serve as the main active components.

Compared with prior art approaches that utilize either deer antler stem cells alone or the conditioned medium alone, the lyophilized powder of the present disclosure provides greater economic efficiency, fully utilizes both the cells and all extracellular secreted bioactive components, and can be produced with greater simplicity and convenience than conventional cell extracts, while retaining the biological efficacy inherent to deer antler stem cells.

### BRIEF DESCRIPTION OF THE DRAWINGS

FIG. 1A illustrates the morphology of deer antler stem cells according to the present disclosure.

FIG. 1B illustrates the flow cytometric analysis results of the deer antler stem cells according to the present disclosure.

FIG. 2 illustrates a schematic flow diagram showing the production process of the lyophilized stem powder of a cervid animal according to the present disclosure.

FIG. 3 illustrates the results of amino acid composition analysis of the lyophilized stem cell powder according to the present disclosure.

FIG. 4 illustrates the test results of antioxidant activity of the lyophilized stem cell powder of deer antler stem cells according to the present disclosure.

FIG. 5 illustrates the test results of anti-inflammatory activity in PBMCs of the lyophilized stem cell powder of deer antler stem cells according to the present disclosure.

FIG. 6 illustrates the test results of osteogenic differentiation of the lyophilized stem cell powder according to the present disclosure.

### DETAILED DESCRIPTION

The following embodiments are provided to describe the present disclosure in greater detail. Those skilled in the art, upon reading the disclosure of the present specification, will readily understand the advantages and effects of the present disclosure, and may also implement or apply the disclosure in other various embodiments. Therefore, the following embodiments may be modified and/or altered without departing from the scope of the different aspects and applications of the present disclosure, and any element or method within the scope of the present disclosure may be combined with any other element or method disclosed in any embodiment herein.

As used herein, the articles “a,” “an,” and “the” refer to one or more than one (i.e., at least one) grammatical object. The terms “or” and “and/or” are used interchangeably unless otherwise clearly indicated. As used herein, the term “about” refers to typical tolerances within the relevant technical field. For example, “about” may be understood as within approximately two standard deviations from the mean value. When “about” precedes a series of numbers or a numerical range, it is to be understood that “about” modifies each individual number or value within that series or range. For instance, a value is intended to encompass variations of +20%, +10%, +5%, +1%, +0.5%, or +0.1%. As used herein, numerical ranges are inclusive and combinable, and any numerical value falling within a stated range can be treated as either a minimum or maximum value from which sub-ranges can be derived.

For example, a numerical range of “20% to 30%” is intended to include any sub-range within that range, such as from 20% to 25%, from 25% to 30%, or from 22.5% to 27.5%. Such variations in values may result, for example, from experimental error, typical variation in the measurement or handling of manufactured compounds, compositions, concentrates, or formulations, differences in the source, manufacture, or purity of the starting materials or ingredients used in the present disclosure, or similar considerations.

As used herein, the term “administration” refers to any technique or route by which an active ingredient (for example, the stem cell lyophilized powder or the stem cell lyophilized powder composition) is delivered to a subject systemically or locally, such that at least a portion of the active ingredient is localized at a desired site to produce the intended effect. For instance, the active ingredient of the present disclosure may be administered orally to a subject; however, the disclosure is not limited thereto. When an effective amount of the present disclosure is administered via the gastrointestinal tract, it may typically be formulated into a unit dosage form (for example, a tablet), though the disclosure is not limited in this respect.

As used herein, the term “subject” refers to any vertebrate, including but not limited to humans or mammals such as deer, mule, elk, or black-tailed deer. In certain preferred embodiments, the subject is a mammal, such as a human or a non-human mammal, including domesticated mammals such as dogs, cats, horses, rats, or mice, or livestock mammals such as cattle, sheep, pigs, or deer.

The term “comprise” or “comprising,” as used herein, is intended to mean “including but not limited to,” and may be used interchangeably therewith. For example, when a claim recites that a target “comprises” a limitation, unless otherwise stated, it may additionally include other ingredients, elements, components, structures, regions, portions, devices, systems, steps, or connections, and does not exclude additional limitations.

As used herein, the term “effective amount” refers to an amount of an active agent or pharmaceutical composition that is sufficient to produce a desired effect in a subject in need thereof. For example, a “cosmetically effective amount” refers to an amount of a composition administered at a specific dosage sufficient to achieve the desired appearance, feel, and/or protective effect.

In certain embodiments, an effective amount of the composition prevents or reduces the appearance and/or symptoms associated with an undesired condition. The effective amount may vary as determined by one skilled in the art depending on factors such as the use of excipients, route of administration, potential combination with other therapeutic treatments, or the condition to be treated, without limiting the scope of the present disclosure.

As used herein, the term “cosmetically or pharmaceutically acceptable carrier” refers to a pharmaceutically acceptable material, medium, or composition such as a solid or liquid filler, binder, diluent, preservative, biocompatible solvent, disintegrant, lubricant, suspending agent, flavoring agent, capsule material, thickener, acid, surfactant, chelating agent, wetting agent, or any combination thereof. In certain embodiments, each component being “pharmaceutically acceptable” means that it is compatible with other ingredients of cosmetic or pharmaceutical formulations and suitable for contact with organs or tissues of a subject (e.g., a mammal) without producing excessive toxicity, allergic reactions, irritation, immunogenicity, or other adverse complications. References describing suitable carriers include Remington: The Science and Practice of Pharmacy, 22nd ed., Allen, Ed., Philadelphia, PA, 2012; Handbook of Pharmaceutical Excipients, 7th ed., Rowe et al., Eds., The Pharmaceutical Press and the American Pharmaceutical Association, 2012; Handbook of Pharmaceutical Additives, 3rd ed., Ash and Ash, Eds., Gower Publishing Company, 2007; and Pharmaceutical Preformulation and Formulation, 2nd ed., Gibson, Ed., CRC Press LLC, Boca Raton, FL, 2009.

As used herein, the term “carrier” may refer to a solid, solution, or mixture in which stem cells and/or their derivatives are suspended for use in topical treatment procedures, transplantation, or other subsequent applications. The carrier includes, but is not limited to, gels, ointments, pastes, or aerosol sprays.

As used herein, the term “cosmetically or pharmaceutically acceptable bulking agent” refers to a reagent that, during lyophilization and drying processes, prevents denaturation of the active ingredient, inhibits sublimation of the active component along with water vapor, facilitates formation of the active substance into a solid matrix, and prevents oxidative degradation during freeze-drying or storage. Examples include maltodextrin, mannitol, trehalose, glycine, glucose, sucrose, lactose, dextran, polyvinylpyrrolidone (PVP), glycerol, dimethyl sulfoxide (DMSO), and gelatin. In at least one embodiment, the cosmetically or pharmaceutically acceptable bulking agent comprises trehalose and maltodextrin. In at least one embodiment, the lyophilized powder of deer antler stem cells according to the present disclosure comprises 4-15% trehalose and 5-20% maltodextrin.

It has been discovered that administration of the lyophilized powder derived from deer antler stem cells to a subject in need thereof can promote at least one of osteogenic differentiation, anti-inflammatory activity, or antioxidant activity, thereby restoring or enhancing corresponding physiological functions.

As used herein, the term “stem cells of a cervid animal” refers to stem cells derived from deer antler, umbilical cord, placenta, adipose tissue, bone marrow, or amniotic fluid of a deer, but is not limited thereto. Stem cells are capable of self-renewal and differentiation into at least two distinct cell lineages. Furthermore, as used herein, throughout the life cycle of an organism, “stem cells” also refer to cells possessing indefinite replicative capacity that, under certain conditions or in response to specific signals, are capable of differentiating into multiple types of cells constituting the organism. In other words, stem cells have the potential to develop into mature cells characterized by distinct morphology and specialized biological functions.

As used herein, the term “whole cell components” refers to materials containing all cellular constituents, including the nucleus, cytoplasm, cell membrane, and all components within various organelles, such as soluble and insoluble proteins, lipids, nucleic acids, carbohydrates, and biomolecular complexes. A whole-cell lyophilized powder containing “whole cell components” retains all intracellular constituents of the cell. This is distinct from commonly known cell extracts or cell lysates, which are typically prepared by extracting specific substances or proteins using a defined extraction buffer or lysis solution, followed by removal of cell debris and other insoluble materials prior to lyophilization. Accordingly, lyophilized powders prepared from cell extracts or lysates differ in composition from the whole-cell lyophilized powder described herein, which contains all cellular components in their entirety.

As used herein, the terms “medium,” “basal medium,” or “media” refer to compositions used for microbial growth or cell culture. Different types of media may be employed to culture different types of cells. A medium suitable for stem cell growth may contain essential nutrients such as amino acids, salts (e.g., ferric nitrate nonahydrate, potassium chloride, magnesium sulfate, sodium chloride, sodium phosphate monobasic), vitamins (e.g., ascorbic acid, folic acid, niacinamide, riboflavin, vitamin B12), and may further include supplements such as iron, glucose, non-essential amino acids, sodium pyruvate, β-mercaptoethanol, L-glutamine, fetal bovine serum (FBS), and leukemia inhibitory factor (LIF). Those skilled in the art can determine an appropriate medium for the initial preparation of stem cells. Commonly used stem cell culture media include, but are not limited to: Iscove's Modified Dulbecco's Medium (IMDM), Dulbecco's Modified Eagle Medium (DMEM), KnockOut™ DMEM (KO-DMEM), DMEM/F12, RPMI 1640, McCoy's 5A Medium, α-Minimum Essential Medium (α-MEM), F-12K Nutrient Mixture (Kaighn's modification), X-Vivo 20, Stemline, CC100, H2000, Stemspan, MCDB 131 Medium, Eagle's Basal Medium (BME), Glasgow's Minimum Essential Medium (GMEM), Modified Eagle's Medium (MEM), Opti-MEM I Reduced Serum Medium, Waymouth's MB 752/1 Medium, Williams' Medium E, NCTC-109 Medium, Neurobasal Medium, BGJb Medium, Brinster's BMOC-3 Medium, CMRL Medium, CO₂-independent Medium, and Leibovitz's L-15 Medium.

As used herein, the term “conditioned medium” refers to a product obtained after culturing specific cells or tissues and subsequently collecting the culture medium. When cells or tissues are cultured in a medium, they may secrete various factors that support or influence the behavior of other cells. Accordingly, a conditioned medium is enriched with the secretome secreted by the cultured cells or tissues, which includes components such as exosomes, extracellular vesicles, and soluble proteins. The composition of the conditioned medium is influenced by multiple factors within the cellular microenvironment. The factors present in the conditioned medium include, but are not limited to, hormones, cytokines, extracellular matrix (ECM) components, proteins, vesicles, antibodies, chemokines, receptors, inhibitors, and particles.

In at least one embodiment of the present disclosure, the lyophilized powder comprises an effective amount of stem cells derived from a cervid animal, wherein the stem cells are obtained from deer antler.

As used herein, the term “cervid animal” refers to animals belonging to the family Cervidae under the order Artiodactyla and the suborder Ruminantia, which may include, but are not limited to, white-tailed deer, mule deer, wapiti (elk), moose, red deer, reindeer (caribou), sika deer, fallow deer, roe deer, sambar deer, and maral deer. In at least one embodiment of the present disclosure, all male deer (except roe deer) and female reindeer bear antlers that are renewed annually.

In at least one embodiment of the present disclosure, the term “deer antler” refers to the unossified, velvet-covered young antler of a male deer (or of female deer in certain species), which is a rare organ among mammals capable of complete periodic regeneration.

In at least one embodiment of the present disclosure, the lyophilized powder derived from deer antler stem cells, or a composition comprising such lyophilized powder, exhibits at least one of the following effects: promotion of osteogenic differentiation, anti-inflammatory activity, and antioxidant activity.

Accordingly, the stem cell lyophilized powder or composition thereof can contribute to promoting osteogenic differentiation, alleviating inflammation, or providing antioxidation, and may assist in preventing or delaying aging or degeneration associated with bone metabolism, inflammation, or oxidative processes.

In at least one embodiment of the present disclosure, the lyophilized powder derived from deer antler stem cells or a composition comprising such powder exhibits efficacy in slowing down, treating, or preventing aging or degeneration associated with bone formation, inflammation, or oxidation.

In at least one embodiment of the present disclosure, the stem cells are derived from deer antler, but the disclosure is not limited thereto. In at least one embodiment, the stem cells are obtained from a cervid animal and cultured for 4 to 14 days for expansion. In certain embodiments, flow cytometry analysis indicates that the deer antler-derived stem cells express at least one of the antigens CD73, CD95, and CD105; in preferred embodiments, the cells express at least two of these antigens; and in more preferred embodiments, the cells express all three antigens, CD73, CD95, and CD105.

In at least one embodiment of the present disclosure, the lyophilized powder of cervid stem cells that is effective in slowing, treating, or preventing aging or degeneration associated with bone differentiation, inflammation, or oxidation may be used alone or in combination with at least one promoting agent that enhances osteogenic differentiation, anti-inflammatory activity, and/or antioxidant activity, thereby achieving therapeutic or preventive effects for medical conditions related to bone, inflammatory, or oxidative degeneration.

### EXAMPLES

The exemplary embodiments of the present disclosure are further described in the following examples. These examples are provided for illustrative purposes only and should not be construed as limiting the scope of the present disclosure in any way.

### Example 1—Collection of Deer Antler Stem Cells and Preparation of a Master Cell Bank (MCB)

### A. Collection of Deer Antler Stem Cells

Deer antler tissues were collected from male deer aged between 2 and 15 years (Formosan sambar deer or Formosan sika deer), the apical portion of deer antler tissues were collected, the tissue of apical portion are still in their early growth stage, approximately 10 to 70 days after initiation of new growth. The apical portion of the antler was excised, cut into small blocks, and immersed in 70% ethanol for approximately 1 to 2 minutes for surface disinfection. The antler was then transversely sectioned, and about 3 to 5 cm of the apical tissue was retained and further subdivided. The subdivided blocks were again immersed in 70% ethanol for about 1 to 2 minutes for disinfection, after which the epidermal layer was removed. The topmost 1 to 5 mm cartilage tissue of the antler tip was excised, minced into small fragments of approximately 1 cubic millimeter, and digested with collagenase at 37° C. for 1 to 12 hours.

The digested material was passed through a 40-100 μm pore-size filter to remove undigested tissue fragments. The filtrate was then centrifuged, and the resulting cell pellet was resuspended in Dulbecco's Modified Eagle Medium (DMEM) supplemented with 10-20% fetal bovine serum (FBS). The cells were counted and seeded into appropriate culture vessels. After several days of culture, multiple cell colonies were formed. When cell confluence reached greater than 70%, or after approximately one week of culture, the cells were detached using TrypLE enzyme solution. The cells obtained at this stage were designated as passage 0 (P0) and subsequently reseeded into new culture vessels as passage 1 (P1).

When cell reached approximately 80-90% confluence, the culture was further subcultured. At passage 2 (P2) or passage 3 (P3), the cells were cryopreserved to establish the master cell bank (MCB).

### B. Cell Characterization and Analysis

Cells from the master cell bank (MCB) were thawed and cultured in a T175 flask until cell exceeded 70% confluence. The cells were then detached using an enzyme solution and subjected to surface antigen staining with antibodies against CD73, CD90, and CD105. Surface antigen expression was analyzed by flow cytometry.

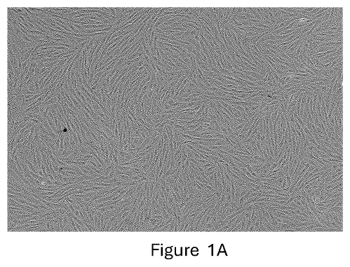

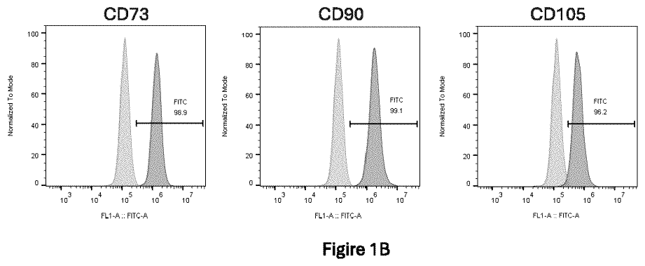

FIG. 1A illustrates the morphology of the deer antler stem cells, and FIG. 1B shows the results of the surface antigen analysis by flow cytometry. The deer antler stem cells expressed surface antigens CD73, CD90, and CD105. The FITC label shown in FIG. 1B refers to fluorescein isothiocyanate.

As shown in FIG. 1A, microscopic observation revealed that the cultured cells derived from deer antler exhibited a fibroblast-like morphology, demonstrating the typical appearance of the deer antler stem cells under a microscope.

### Example 2—Culture of Deer Antler Stem Cell and Collection of Cell and Conditioned Medium (CM)

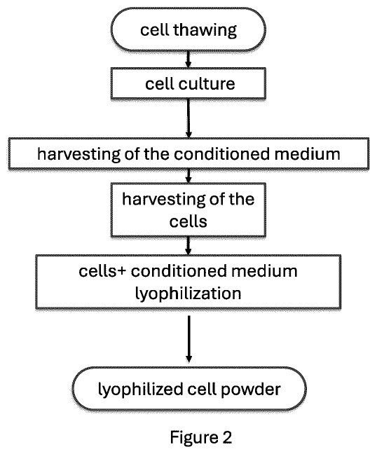

As illustrated in FIG. 2, the production process of the lyophilized powder of deer antler stem cells according to the present disclosure may include at least the following steps: cell thawing; cell culture; harvesting of the conditioned medium; harvesting of the cells; mixing of the cells with the conditioned medium; lyophilization; and preparation of the lyophilized cell powder.

First, a conditioned medium from deer antler stem cells was collected. 1×10⁷ cells from the deer antler stem cell master cell bank (MCB) was thawed in a 37° C. water bath, resuspended in Dulbecco's Modified Eagle Medium (DMEM) supplemented with 10 to 20% fetal bovine serum (FBS), and seeded into a HyperFlask (T175, 10-layer). The culture was maintained at 37° C. in a humidified atmosphere containing 5% CO₂.

The conditioned medium was harvested on days 3, 6, and 9 of culture and stored at −80° C. Each time the conditioned medium was collected, it was replaced with fresh DMEM containing 10 to 20% FBS.

On day 12 of culture, the final conditioned medium was collected. The cells were gently rinsed with an appropriate volume of phosphate-buffered saline (PBS). The adherent cells were detached from the HyperFlask using an enzymatic solution, and the resulting cell suspension was collected. After centrifugation to remove residual enzyme, the cell pellet was resuspended in the conditioned medium collected on the same day and stored at −80° C. (as shown in FIG. 2).

### Example 3—Preparation of Lyophilized Powder

### Experimental Group: Preparation of the Lyophilized Stem Cell Powder of a Cervid Animal

The collected deer antler stem cells and conditioned medium were mixed, subjected to freeze-drying, and pulverized to obtain a lyophilized stem cell powder of a cervid animal, which comprised a lyophilized powder of deer antler stem cells and a lyophilized powder of the conditioned medium. The lyophilized powder of the cervid animal contained a whole-cell lyophilizate including all cellular components, and the conditioned medium was the medium obtained after culturing the deer antler stem cells.

For example, the conditioned media collected on days 3, 6, and 9 (which did not contain deer antler stem cells) and the mixture collected on day 12 (containing both deer antler stem cells and conditioned medium) were thawed at 4° C. Trehalose (8 wt %) and maltodextrin (10 wt %) were added and mixed thoroughly until completely dissolved to form a homogeneous sample. The following steps were then performed to obtain the lyophilized stem cell powder of a cervid animal:

### (1) Lyophilization Process

The prepared samples were pre-cooled at −40° C. for 240 minutes, followed by condensation at −40° C. for 2 minutes. Vacuum pressure was reduced to 0.2 torr, and the temperature was gradually increased to 30° C. Sublimation was carried out for 24 to 60 hours to form a lyophilized product containing the deer antler stem cells and the conditioned medium.

### (2) Pulverization Process

The lyophilized product obtained above was pulverized using a vertical grinder and sieved through a 0.6 mm mesh. The processing capacity was approximately 1 kg per 10 minutes, yielding the lyophilized stem cell powder of a cervid animal.

The resulting lyophilized stem cell powder of a cervid animal was reconstituted in sterile deionized water and stained with Trypan Blue for cell viability assessment. The observed cell viability was 0%, indicating that the lyophilized stem cell powder of a cervid animal contained no viable cells.

### Control Group: Preparation of Conventional Deer Antler Powder

Dried deer antlers were directly pulverized into powder, and soluble proteins were extracted using RIPA cell lysis buffer.

### Example 4—Component Analysis of the Lyophilized Stem Cell Powder of a Cervid Animal Determination of Protein Content

The protein content of the experimental group (hereinafter referred to as the “stem cell lyophilized powder”) prepared according to Example 3 and the control group (hereinafter referred to as the “deer antler powder”) was quantified using the bicinchoninic acid (BCA) assay.

Table 1 summarizes the data from four batches of the stem cell lyophilized powder and the deer antler powder.

The protein content of the stem cell lyophilized powder prepared according to the present disclosure ranged from approximately 207.4 mg/g to 329 mg/g, which was about 1-4 fold higher than that of the deer antler powder.

Based on statistical analysis of the four batches, the possible range of protein content in the stem cell lyophilized powder prepared according to the present disclosure was from 101.2 mg/g (based batch 3 value minus two standard deviations) to 435.2 mg/g (based on batch 4 value plus two standard deviations), calculated based on the total weight of the stem cell lyophilized powder. The possible range of protein content in the stem cell lyophilized powder prepared according to the present disclosure was from 145.8 mg/g to 358.2 mg/g (based on mean±2 standard deviations).

TABLE 1
Protein content of stem cell lyophilized
powder and deer antler powder

| | Protein | | |
|---|---|---|---|
| | content | Mean | |
| Batch | (mg/g) | (mg/g) | SD |
| stem cell lyophilized powder | | | |
| 1 | 233.3 | 252 | 53.1 |
| 2 | 238.2 | | |
| 3 | 207.4 | | |
| 4 | 329 | | |
| deer antler powder | 112.9 | N/A | N/A |

### Determination of Free Amino Acids

The stem cell lyophilized powder prepared according to Example 3 and the deer antler powder were each reconstituted in sterile deionized water. The free amino acid content was analyzed in accordance with the CNS-12632 analytical method for fruit and vegetable juice beverages.

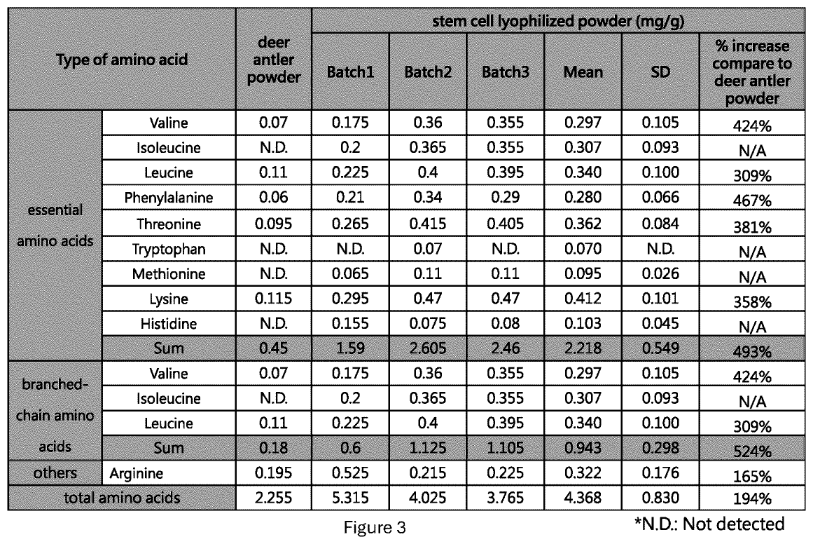

FIG. 3 shows the identification results of free amino acids from three independent batches. As illustrated in FIG. 3, the stem cell lyophilized powder had an average total amino acid content of 4.368 mg/g, an average essential amino acid content of 2.218 mg/g, and an average branched-chain amino acid content of 0.943 mg/g. Based on statistical analysis of three batches, the quantitative data for total amino acids, essential amino acids, and branched-chain amino acids in the stem cell lyophilized powder are summarized in Table 2.

Accordingly, the possible ranges of amino acid contents in the stem cell lyophilized powder prepared according to the present disclosure were as in Table 2 (based on mean±2 standard deviations), calculated with respect to the total weight of the stem cell lyophilized powder.

TABLE 2
Range of amino acids content in stem cell lyophilized powder

| | Mean − | Mean + | Range of |
|---|---|---|---|
| Item | 2*SD | 2*SD | Content |
| total amino acids | 2.71 | 6.03 | 2.71 to 6.03 |
| essential amino acids | 1.12 | 3.32 | 1.12 to 3.32 |
| branched-chain amino acids | 0.35 | 1.54 | 0.35 to 1.54 |

Compared with the conventional deer antler powder, the stem cell lyophilized powder exhibited approximately 1.9-fold higher total amino acid content, 4.9-fold higher essential amino acid content, and 5.2-fold higher total branched-chain amino acid content.

### Example 5—Analysis of Activin Content in the Stem Cell Lyophilized Powder

### Determination of Activin Content

The stem cell lyophilized powder prepared according to Example 3 was reconstituted in an appropriate volume of deionized water. The content of activin was determined using a commercially available ELISA kit (RayBiotech).

Table 3 shows the activin content data obtained from three batches of the stem cell lyophilized powder.

TABLE 3
Activin Content in Lyophilized Stem Cell Powder

| Batch | Activin Content (ng/g) | Mean (ng/g) | Standard Deviation |
|---|---|---|---|
| 1 | 43.04 | 83.7 | 35.8 |
| 2 | 97.82 | | |
| 3 | 110.36 | | |

The activin content of the stem cell lyophilized powder prepared according to the present disclosure ranged from approximately 43 ng/g±35.8 ng/g (based batch 1 value minus/plus standard deviations) to 110 ng/g±35.8 ng/g (based batch 3 value minus/plus standard deviations). Based on statistical analysis of the three batches, the possible activin content in different batches of the stem cell lyophilized powder was estimated to range from 12.1 ng/g to 155.3 ng/g (based on mean±2 standard deviations), calculated with respect to the total weight of the stem cell lyophilized powder.

### Example 6—Preparation of Lyophilized Powder Compositions for Promoting Osteogenic Differentiation, Anti-Inflammation, or Antioxidation

The lyophilized powder composition prepared according to the embodiments of the present disclosure comprises a lyophilized stem cell powder of a cervid animal, at least one promoting agent that enhances osteogenic differentiation, anti-inflammatory activity, or antioxidant activity, and, optionally, a pharmaceutically or cosmetically acceptable carrier.

In at least one embodiment of the present disclosure, the promoting agent may include at least one selected from the group consisting of Cistanche tubulosa extract, turmeric extract, organic maca, arginine, cranberry extract, Cordyceps sinensis mycelium, and apple-grape extract.

In certain embodiments, the components of the lyophilized powder composition are as shown in Table 4 or Table 5 below.

TABLE 4
Example Composition of Lyophilized Powder (Formulation I)

| | | Content | |
|---|---|---|---|
| No. | Component | (%) | Function/Effect |
| 1 | Lyophilized | 20-99 | Anti-inflammatory, antioxidant, |
| | stem cell | | enhances bone development, promotes |
| | powder of a | | wound healing |
| | cervid animal | | |
| 2 | Cistanche | 0-20 | Anti-inflammatory, immune |
| | tubulosa | | enhancement, neuroprotection, bone |
| | extract | | strengthening, vasodilation |
| 3 | Turmeric | 0-20 | Promotes metabolism, antioxidant |
| | extract | | activity |
| 4 | Organic maca | 0-20 | Enhances athletic performance, |
| | (Peruvian) | | improves male reproductive function, |
| | | | alleviates sexual dysfunction |
| 5 | Arginine | 0-20 | Promotes metabolism, nitric oxide |
| | | | production, and protein/energy synthesis |

TABLE 5
Example Composition of Lyophilized Powder (Formulation II)

| | | Content | |
|---|---|---|---|
| No. | Component | (%) | Function/Effect |
| 1 | Lyophilized stem cell | 10-99 | Anti-inflammatory, antioxidant, |
| | powder of a cervid | | enhances bone development, |
| | animal | | promotes wound healing |
| 2 | Cranberry extract | 0-50 | Antioxidant, cardiovascular |
| | | | protection, skin repair, promotes |
| | | | metabolism, prevents urinary |
| | | | tract infection |
| 3 | Cordyceps sinensis | 0-25 | Enhances exercise endurance, |
| | mycelium | | delays fatigue, boosts immunity, |
| | | | improves sleep quality |
| 4 | Vinitrox ™ | 0-15 | Supports metabolism, improves |
| | polyphenols (apple- | | complexion, antioxidant, |
| | grape extract) | | regulates digestive function |

### Example 7—Application of the Lyophilized Stem Cell Powder of a Cervid Animal

The lyophilized stem cell powder of a cervid animal prepared according to the embodiments of the present disclosure can be used for preparing a lyophilized powder composition that promotes osteogenic differentiation, anti-inflammatory activity, or antioxidant activity.

In at least one embodiment of the present disclosure, the lyophilized powder composition further comprises a promoting agent that enhances osteogenic differentiation, anti-inflammatory activity, and/or antioxidant activity, together with a pharmaceutically or cosmetically acceptable carrier as required.

In at least one embodiment, the promoting agent may include at least one selected from the group consisting of Cistanche tubulosa extract, turmeric extract, organic maca, arginine, cranberry extract, Cordyceps sinensis mycelium, and apple-grape extract.

In some embodiments, the components of the lyophilized powder composition are as shown in Table 4 or Table 5 above.

### Example 8—Antioxidant Activity Assay

### Antioxidant Assay Using Human Monocytic THP-1 Cells

The stem cell lyophilized powder prepared in Example 3 was reconstituted with sterile deionized water and sterilized by filtration through a 0.22 μm membrane filter.

THP-1 cells were cultured in 6-well plates for 24 hours to allow cell adhesion. The reconstituted stem cell lyophilized powder samples were added to the cultures and incubated for 1 hour, followed by sequential addition of approximately 10 μM DCFH-DA (2′,7′-dichlorodihydrofluorescein diacetate) dye and 50-100 μM hydrogen peroxide (H₂O₂) to induce oxidative stress in the cells.

After completion of the reaction, the culture medium was removed, and the cells were washed twice with phosphate-buffered saline (PBS). The cells were then treated with trypsin for 5 minutes, collected, and transferred into centrifuge tubes containing fresh culture medium. The cells were centrifuged at 400×g for 5 minutes and washed with PBS (the entire process was performed in the dark). The cells were resuspended in PBS and analyzed by flow cytometry.

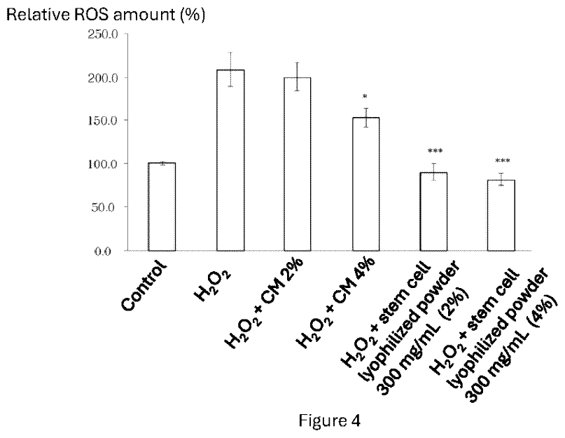

Fluorescence was detected with excitation at 450-490 nm to quantify green fluorescence intensity. The experimental results are summarized in FIG. 4.

As shown in FIG. 4, the stem cell lyophilized powder exhibited greater antioxidant capacity than the deer antler conditioned medium (CM). Compared with the control group, the difference was statistically significant (p<0.05; **p<0.001). In FIG. 4, “ROS” denotes reactive oxygen species.

### Example 9—Anti-Inflammatory Assay Using Peripheral Blood Mononuclear Cells (PBMCs) PBMC Anti-Inflammatory Test

The stem cell lyophilized powder prepared according to Example 3 was reconstituted in sterile deionized water and sterilized using a 0.22 μm membrane filter.

PBMCs were co-cultured with the reconstituted stem cell lyophilized powder samples and subsequently stimulated with lipopolysaccharide (LPS) to induce an inflammatory response. Samples were collected after 6 hours and 24 hours of induction, and total mRNA was extracted. The relative mRNA expression levels of 13 cytokines—IL1B (IL1B), IL8, IL6, IL10, IL18, TNFα (TNFA), IL16, IL23, IL12A, IFNG, TGFβ (TGFB), IL3, and IL4—were quantified by RT-qPCR analysis.

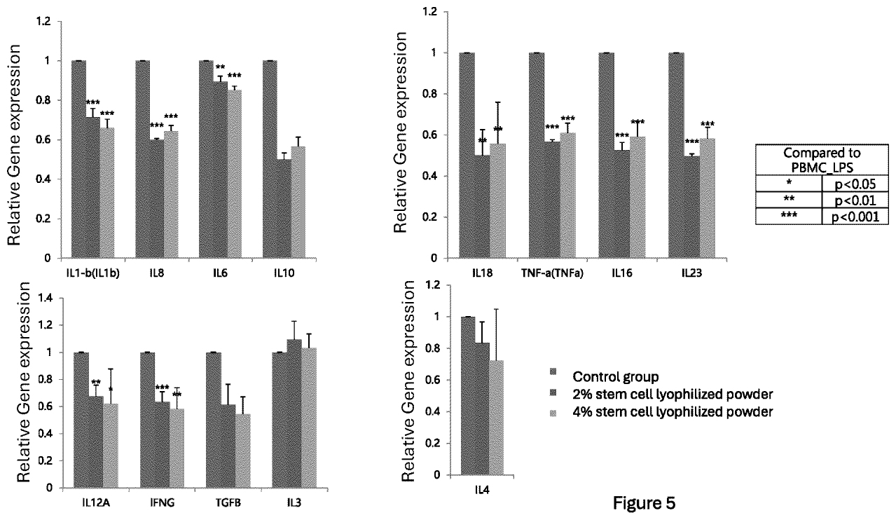

FIG. 5 shows the relative mRNA expression results obtained after 6 hours of LPS induction following treatment with 2% and 4% concentrations of the stem cell lyophilized powder. The results indicate that the stem cell lyophilized powder effectively suppressed the expression of multiple inflammatory cytokines, demonstrating a strong anti-inflammatory effect (p<0.05; *p<0.01; **p<0.001, compared with the control group PBMC_LPS).

### Example 10—Osteogenic Differentiation Assay

### Osteogenic Differentiation Potential of Adipose-Derived Mesenchymal Stem Cells (AD-MSCs)

The stem cell lyophilized powder prepared according to Example 3 was reconstituted in sterile deionized water and sterilized using a 0.22 μm membrane filter.

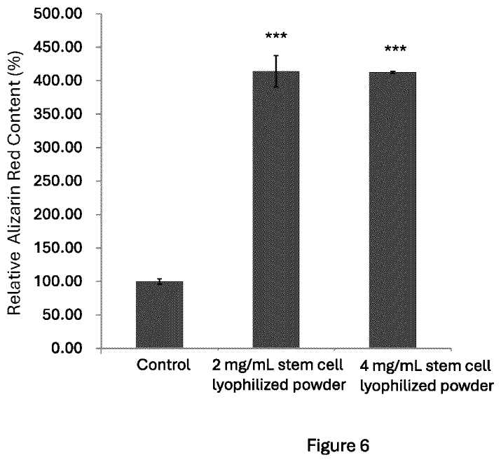

AD-MSCs were seeded at a density of 15,000 cells/cm². After 2-3 days, when the cells reached confluence, the medium was replaced with an osteogenic differentiation medium composed of αMEM supplemented with 100 nM dexamethasone, 200 μM ascorbic acid, and 10 mM glycerol 2-phosphate. The reconstituted stem cell lyophilized powder samples were added concurrently. The culture medium was replaced twice per week. After 7-14 days of differentiation, calcium deposition was visualized by Alizarin Red S staining, and the stained dye was subsequently eluted for quantitative measurement, as shown in FIG. 6.

The results in FIG. 6 demonstrate that the stem cell lyophilized powder promoted osteogenic differentiation of AD-MSCs.

The foregoing detailed description of the specific embodiments is provided for illustrative purposes to demonstrate the manner in which the present disclosure may be practiced and is not intended to limit the scope of the present disclosure. All modifications and variations that can be conceived by those skilled in the art based on the teachings herein shall fall within the scope of the appended claims of the present disclosure.

What is claimed is:

1. A lyophilized stem cell powder of a cervid animal, comprising:
(a) a lyophilized powder of a deer antler stem cell; and
(b) a lyophilized powder of a conditioned medium; wherein the lyophilized powder of the deer antler stem cell comprise a whole-cell lyophilized powder containing whole cellular components, and the conditioned medium is a conditioned medium obtained after culturing the deer antler stem cell.

2. The lyophilized stem cell powder of claim 1, wherein the deer antler stem cell expresses at least one antigen selected from the group consisting of CD73, CD90, and CD105.

3. The lyophilized stem cell powder of claim 1, wherein the protein concentration of the lyophilized stem cell powder is about 101.2 mg/g to 435.2 mg/g.

4. The lyophilized stem cell powder of claim 1, wherein the lyophilized stem cell powder comprises:
(a) about 1.12 mg/g to 3.32 mg/g of essential amino acids;
(b) about 0.35 mg/g to 1.54 mg/g of branched-chain amino acids; or
(c) about 2.71 mg/g to 6.03 mg/g of total amino acids.

5. The lyophilized stem cell powder of claim 1, wherein activin concentration of the lyophilized stem cell powder is about 12.1 ng/g to 155.3 ng/g.

6. The lyophilized stem cell powder of claim 1, wherein no viable cells are present in the lyophilized stem cell powder.

7. The lyophilized stem cell powder of claim 1, further comprising a pharmaceutically or cosmetically acceptable bulking agent.

8. A lyophilized stem powder composition comprising:
(a) the lyophilized stem cell powder of claim 1; and
(b) an agent that enhances at least one activity selected from the group consisting of osteogenic differentiation, anti-inflammatory activity, and antioxidant activity.

9. A method for preparing the lyophilized stem cell powder of claim 1, comprising:
(a) culturing the deer antler stem cell in a culture medium to obtain a conditioned medium;
(b) collecting the conditioned medium;
(c) collecting the cultured deer antler stem cells; and
(d) freeze-drying and pulverization the collected conditioned medium and the collected deer antler stem cells to obtain the lyophilized stem cell powder of the cervid animal.

10. The method of claim 9, wherein the method further comprising mixing the collected conditioned medium and the collected deer antler stem cells prior to the freeze-drying and pulverization process.

11. The method of claim 10, wherein the freeze-drying process to obtain the lyophilized stem cell powder comprising the following procedure:
(a) condensing the mixture of the conditioned medium and the deer antler stem cells at −40° C.,
(b) vacuuming the mixture e to a pressure of 0.2 torr,
(c) heating the mixture to 30° C., and
(d) sublimating the mixture.

12. The method of claim 11, wherein the pulverization process to obtain the lyophilized stem cell powder comprising the following procedure:
(a) pulverized using a pulverizer, and
(b) and sieved through a 0.6 mm mesh.

13. The method of claim 9, wherein the conditioned medium is a DMEM culture medium.

14. The method of claim 9, wherein no viable cells are present in the lyophilized stem cell powder.

15. A use of the lyophilized stem cell powder of claim 1 for the preparation of a lyophilized powder composition that promotes at least one activity selected from the group consisting of osteogenic differentiation, anti-inflammatory activity, and antioxidant activity.
